(19) United States
(12) Patent Application Publication
(10) Pub. No.: US 2025/0180539 A1
(43) Pub. Date: Jun. 5, 2025

(54) Soil Analysis Methods, Systems and Kits

(71) Applicant: Precision Planting LLC, Tremont, IL (US)

(72) Inventor: Tamara Fitzjarrald, Canton, IL (US)

(21) Appl. No.: 18/843,887

(22) PCT Filed: Feb. 2, 2023
(86) PCT No.: PCT/IB2023/050901

§ 371 Date: Sep. 4, 2024

Related U.S. Application Data
(60) Provisional application No. 63/269,064, filed on Mar. 9, 2022.
(60) Provisional application No. 63/269,060, filed on Mar. 9, 2022.

(51) Int. Cl. G01N 33/24; G01N 21/31; G01N 31/02; G01N 31/22

(52) U.S. Cl. CPC G01N 33/245; G01N 21/31; G01N 31/02; G01N 31/22

(57) ABSTRACT

A method for quantifying the magnesium ion amount in a soil sample, the method including: extracting calcium ions from a soil sample to produce a soil sample substantially free of calcium: mixing the substantially free calcium soil sample with ethylene glycol-bis(β-aminoethyl ether)-N,N,N′,N′-tetraacetic acid tetrasodium salt (EGTA) to produce a first sample; mixing the first sample with an indicator reagent to produce a second sample; and determining the magnesium ion amount of the second sample; wherein the indicator reagent comprises o-cresolphthalein complexone (OCPC) and tetrabutyl ammonium hydroxide (TBAH). Also, a system and a kit using the method.

### CROSS REFERENCE TO RELATED APPLICATIONS

This application claims priority to U.S. Provisional Application Nos. 63/269,060, filed 9 Mar. 2022 and 63/269,064, filed 9 Mar. 2022, the contents of each are incorporated herein by reference in their entireties.

### BACKGROUND

Soil analysis of agricultural fields allows a grower to know whether there are sufficient amounts of nutrients in the soil for planting. For example, if one or more nutrients is deficient, then the nutrient(s) can be supplemented to the soil. There are many standardized soil tests available today, such as measurement of pH with a pH meter and measurement of soil nutrients by atomic spectroscopy. However, these tests were designed for laboratory testing, and as such, are not suitable for soil sampling within the field.

More so, while quantification of ions, such as calcium and magnesium, may be effectively performed using laboratory instrumentation, such as an ICP, use of colorimetric methods for accurate analysis is challenging due to similarities of the chemical properties and reactivity of the two ions. As such, correlating the results towards one ion or the other is difficult.

Therefore, it would be desirable to be able to test soil samples “on the go” with soil tests that can provide accurate results while in the field. There is a need for methods and compositions which may be used to analyze soil samples accurately and efficiently without the need for heavy laboratory equipment.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Applicants have discovered that utilization of certain components within certain methods provide for an accurate analysis of soil samples while in the field. In some aspects, such compositions and methods allow for efficient extraction of calcium and magnesium ions from soil samples. In further aspects, such compositions and methods allow for efficient quantitation of magnesium ions within soil samples.

Thus, in one aspect, the invention provides a method of extracting calcium ions from soil comprising mixing soil with a solvent to produce a soil slurry; mixing the soil slurry with ammonium acetate to produce an extracted sample; filtering the extracted sample to produce a first filtrate; mixing the first filtrate with a precipitation reagent to produce precipitated calcium oxalate and a liquid solution; and separating the calcium oxalate from the liquid solution; wherein the precipitation reagent comprises from about 0.15 to about 0.35 M of an oxalate salt dissolved in a buffer having a pH from about 7.1 or more. In certain embodiments, the solvent is water. In certain embodiments, the volume ratio of soil to solvent used to produce the soil slurry is from about 1:2.5 to about 1:3.5 or from about 1:2.9 to about 1:3.1. In certain embodiments, the concentration of ammonium acetate is from about 0.5 to about 1.5 M, from about 0.6 to about 1.4 M, or from about 0.75 to about 1.25 M. In certain embodiments, the volume ratio of soil slurry to ammonium acetate used to produce the extracted sample is from about 1:1 to about 1:5 or from about 1:2 to about 1:4. In certain embodiments, the volume ratio of first filtrate to precipitation reagent used to produce the precipitated calcium oxalate and a liquid solution is from about 20:1 to about 5:1 or from about 15:1 to about 7:1. In certain embodiments, the oxalate salt is selected from sodium oxalate, potassium oxalate, tin oxalate, naftidrofuryl oxalate, ammonium oxalate, escitalopram oxalate, cesium oxalate, lanthanum oxalate, cerium oxalate, and a mixture thereof. In certain embodiments, the concentration of the oxalate salt is from about 0.28 to about 0.32 or about 0.3 M. In certain embodiments, the oxalate salt is dissolved in buffer comprising boric acid, tetrabutyl ammonium hydroxide, a carbonate, a phosphate, or a mixture thereof. In certain embodiments, the oxalate salt is dissolved in buffer comprising boric acid and tetrabutyl ammonium hydroxide. In certain embodiments, the concentration of non-oxalate components is from about 0.15 to about 0.35 M. In certain embodiments, the pH of the buffer is from about 7.1 to about 8.0 or from about 7.18 to about 7.4. In certain embodiments, the separation of calcium oxalate from liquid solution is performed by filtration. In certain embodiments, the filtration is performed using a filter having a pore diameter of less than about 2 μm. In certain embodiments, the filtration is performed using a filter having a pore diameter of about 1 μm or less. In certain embodiments, the separation of calcium oxalate from liquid solution is performed by centrifugation. In certain embodiments, the separation of calcium oxalate from liquid solution is performed by centrifugation and filtration. In certain embodiments, the invention is a system capable of extracting calcium ions from soil using any one of the methods as described above, the system comprising means to mix soil and solvent to produce a soil slurry; means to mix soil slurry with ammonium acetate to produce an extracted sample; means to filter the extracted sample to produce a first filtrate; means to mix the first filtrate with a precipitation reagent to produce precipitated calcium oxalate and a liquid solution; and means to separate the calcium oxalate from the liquid solution. In further embodiments, the invention is a kit capable of extracting calcium ions from soil using any one of the methods described above, the system comprising means to mix soil and solvent to produce a soil slurry; means to mix soil slurry with ammonium acetate to produce an extracted sample; means to filter the extracted sample to produce a first filtrate; means to mix the first filtrate with a precipitation reagent to produce precipitated calcium oxalate and a liquid solution; and means to separate the calcium oxalate from the liquid solution.

In other embodiments, the invention is directed towards a method for quantifying the magnesium ion amount in a soil sample, the method comprising extracting calcium ions from a soil sample to produce a soil sample substantially free of calcium; mixing the substantially free calcium soil sample with ethylene glycol-bis(β-aminoethyl ether)-N,N,N′,N′-tetraacetic acid tetrasodium salt (EGTA) to produce a first sample; mixing the first sample with an indicator reagent to produce a second sample; and determining the magnesium ion amount of the second sample; wherein the indicator reagent comprises o-cresolphthalein complexone (OCPC) and tetrabutyl ammonium hydroxide (TBAH). In certain embodiments, the EGTA has a concentration from about 0.125 to about 1.25 mM or from about 0.125 to about 0.3 mM. In certain embodiments, the EGTA is in a buffered solution at a pH from about 9 to about 11, about 9.5 to about 10.5, or about 10. In certain embodiments, the EGTA is in a buffer comprising boric acid, sodium hydroxide, and a salt selected from potassium chloride, sodium chloride, and a mixture thereof. In certain embodiments, the salt is potassium chloride. In certain embodiments, the volume ratio of the substantially free calcium soil sample to EGTA to produce a first sample is from about 1:3 to about 1:7 or from about 1:3 to about 1:5. In certain embodiments, the OCPC is present within the indicator reagent at a concentration from about 0.08 to about 0.16 mM or from about 0.1 to about 0.14 mM. In certain embodiments, the TBAH is present within the indicator reagent at a concentration from about 0.008 to about 0.06 M or from about 0.01 to about 0.02 M. In certain embodiments, the pH of the indicator reagent is about 10. In certain embodiments, the indicator reagent comprises boric acid, sodium hydroxide, and a salt selected from potassium chloride, sodium chloride, and a mixture thereof. In certain embodiments, the salt is potassium chloride. In certain embodiments, the volume ratio of the first sample to indicator reagent to produce a second sample is from about 1:3 to about 1:7 or from about 1:3 to about 1:5. In certain embodiments, the magnesium ion determination is performed using spectrophotometry. In certain embodiments, the determination uses a wavelength from 540 to 585 nm, from 555 to 580 nm, or from 560 to 575 nm. In certain embodiments, the determination uses a wavelength of about 574 nm. In certain embodiments, the calcium ion extraction is performed by comprising the steps of mixing soil with a solvent to produce a soil slurry; mixing the soil slurry with ammonium acetate to produce an extracted sample; filtering the extracted sample to produce a first filtrate; mixing the first filtrate with a precipitation reagent to produce precipitated calcium oxalate and a liquid solution; and separating the calcium oxalate from the liquid solution; wherein the precipitation reagent comprises from about 0.15 to about 0.35 M of an oxalate salt dissolved in a buffer having a pH from about 7.1 or more. In certain embodiments, the solvent is water. In certain embodiments, the volume ratio of soil to solvent used to produce the soil slurry is from about 1:2.5 to about 1:3.5 or from about 1:2.9 to about 1:3.1. In certain embodiments, the concentration of ammonium acetate is from about 0.5 to about 1.5 M, from about 0.6 to about 1.4 M, or from about 0.75 to about 1.25 M. In certain embodiments, the volume ratio of soil slurry to ammonium acetate used to produce the extracted sample is from about 1:1 to about 1:5 or from about 1:2 to about 1:4. In certain embodiments, the volume ratio of first filtrate to precipitation reagent used to produce the precipitated calcium oxalate and a liquid solution is from about 20:1 to about 5:1 or from about 15:1 to about 7:1. In certain embodiments, the oxalate salt is selected from sodium oxalate, potassium oxalate, tin oxalate, naftidrofuryl oxalate, ammonium oxalate, escitalopram oxalate, cesium oxalate, lanthanum oxalate, cerium oxalate, and a mixture thereof. In certain embodiments, the concentration of the oxalate salt is from about 0.28 to about 0.32 or about 0.3 M. In certain embodiments, the oxalate salt is dissolved in buffer comprising boric acid, tetrabutyl ammonium hydroxide, a carbonate, a phosphate, or a mixture thereof. In certain embodiments, the oxalate salt is dissolved in buffer comprising boric acid and tetrabutyl ammonium hydroxide. In certain embodiments, the concentration of non-oxalate components is from about 0.15 to about 0.35 M. In certain embodiments, the pH of the buffer is from about 7.1 to about 8.0 or from about 7.18 to about 7.4. In certain embodiments, the separation of calcium oxalate from liquid solution is performed by filtration. In certain embodiments, the filtration is performed using a filter having a pore diameter of less than about 2 μm. In certain embodiments, the filtration is performed using a filter having a pore diameter of about 1 μm or less. In certain embodiments, the separation of calcium oxalate from liquid solution is performed by centrifugation. In certain embodiments, the separation of calcium oxalate from liquid solution is performed by centrifugation and filtration. In further embodiments, the invention is a system capable of quantifying the magnesium ion amount in a soil sample using any one of the methods as described above, the system comprising means to obtain a soil slurry substantially free from calcium ions: means to mix the soil slurry with EGTA to produce a first sample; means to mix the first sample with an indicator reagent to produce a second sample; and means to determine the magnesium ion amount of the second sample. In further embodiments, the invention is a kit capable of quantifying the magnesium ion amount in a soil sample using any one of the methods as described above, the system comprising means to obtain a soil slurry substantially free from calcium ions; means to mix the soil slurry with EGTA to produce a first sample; means to mix the first sample with an indicator reagent to produce a second sample; and means to determine the magnesium ion amount of the second sample.

Further areas of applicability of the present disclosure will become apparent from the detailed description provided hereinafter. It should be understood that the detailed description and specific examples, while indicating the preferred embodiment of the disclosure, are intended for purposes of illustration only and are not intended to limit the scope of the disclosure.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other applications and methods. It is to be understood that the invention is not limited in its application to the details of any particular embodiment shown. The terminology used herein is for the purpose of description and not to limit the invention, its application, or uses.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, “containing”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range.

Unless otherwise specified, all percentages and amounts expressed herein and elsewhere in the specification should be understood to refer to percentages by weight of the total composition. Reference to a molecule, or to molecules, being present at a “wt. %” refers to the amount of that molecule, or molecules, present in the composition based on the total weight of the composition.

According to the present application, use of the term “about” in conjunction with a numeral value refers to a value that may be +/−5% of that numeral. As used herein, the term “substantially free” is intended to mean an amount less than about 5.0 weight %, less than 3.0 weight %, 1.0 wt. %; preferably less than about 0.5 wt. %, and more preferably less than about 0.25 wt. % of the composition.

As used herein, the terms “substantially free of” when used in relation to a component, or components, may refer to a composition that contains a particular component in an amount of less than 5.0 weight %, less than 3.0 weight %, less than 1.0 weight %, less than 0 1 weight %, less than 0.05 weight %, less than 0.01 weight %, less than 0.005 weight %, or less than 0.0001 weight %, based on a total weight of the composition. As used herein, the terms “free of” when used in relation to a component, or components, may refer to a composition that contains a particular component in an undetectable amount of that component, or components.

As used herein, the term “effective amount” refers to an amount that is effective to elicit the desired response, including the amount of a composition that, when used in a reaction, is sufficient to achieve an effect toward the desired result. The effective amount may vary depending on the composition, the pH, and/or the temperature. The effective amount can include a range of amounts.

Unless defined otherwise, all technical and scientific terms used herein have the same meanings as commonly understood by one of ordinary skill in the art to which this invention belongs. All patents, patent applications, publications, and other references cited or referred to herein are incorporated by reference in their entireties for all purposes. In the event of a conflict in a definition in the present disclosure and that of a cited reference, the present disclosure controls.

The present disclosure is directed towards compositions and methods useful for analyzing ion amounts within soil samples. Thus, in certain embodiments, the present disclosure is directed towards a method of extracting calcium and/or magnesium ions from soil. In other embodiments, the present disclosure is directed towards a method for quantifying the magnesium ion amount in a soil sample.

In certain embodiments, the invention includes a method of extracting calcium and/or magnesium ions from soil comprising mixing soil with a solvent to produce a soil slurry; mixing the soil slurry with a solution comprising ammonium acetate to produce an extracted sample; filtering the extracted sample to produce a first filtrate; mixing the first filtrate with a precipitation reagent to produce precipitated calcium oxalate and a liquid solution; and separating the calcium oxalate from the liquid solution; wherein the precipitation reagent comprises from about 0.1 to about 0.5 M, or from about 0.15 to about 0.35 M, of an oxalate salt dissolved in a buffer having a pH from about 7.1 or more.

The volume ratio of soil and solvent to produce a soil slurry may vary. The volume ratio should be an amount which is effective at diluting the soil to be a soil slurry. In certain embodiments, the volume ratio of soil to solvent used to produce the soil slurry is from about 1:1 to about 1:15-including all ranges in between. In certain embodiments, the volume ratio of soil to solvent is from about 1:1 to about 1:10, from about 1:2 to about 1:8, from about 1:2 to about 1:5, from about 1:2.5 to about 1:3.5, or from about 1:2.9 to about 1:3.1. In some embodiments, the volume ratio of soil to solvent is about 1:3.

The solvent used to mix with the soil may vary. While not being bound to theory, it is believed that since the methods utilize inorganic salts, that organic solvents may not be useful. Furthermore, in certain embodiments, solvents which are free from, or do not contain, calcium or magnesium are preferred. Thus, in certain embodiments, the solvent is selected from water, ethanol, and a mixture thereof. In certain embodiments, the solvent is water.

The amount or concentration of the ammonium acetate may vary. One of skill in the art would appreciate that ammonium acetate is a solid salt at room temperature, therefore, ammonium acetate may be dissolved in a solution for use in the compositions and methods described herein. In certain embodiments, the soil slurry is mixed with a concentrate of ammonium acetate having a concentration of from about 0.5 to about 3.0 M, from 0.5 to about 1.5 M, from 0.5 to about 2.5 M, from 0.5 to about 2.0 M, from about 0.5 to about 1.5 M, from about 0.6 to about 1.4 M, or from about 0.75 to about 1.25 M. In certain embodiments, the concentration of ammonium acetate is about 1.0 M. The ammonium acetate may be dissolved in certain solvents. In certain embodiments, the solvent is water.

The volume ratio of soil slurry to ammonium acetate used to produce the extracted sample may vary. In certain embodiments, the volume ratio of soil slurry to ammonium acetate is from about 1:1 to about 1:15-including all ranges in between. In certain embodiments, the volume ratio of soil to solvent is from about 1:1 to about 1:10, from about 1:2 to about 1:8, from about 1:2 to about 1:5, from about 1:2.5 to about 1:3.5, or from about 1:2.9 to about 1:3.1. In some embodiments, the volume ratio of soil slurry to ammonium acetate used to produce the extracted sample is from about 1:1 to about 1:5, from about 1:2 to about 1:4, or about 1:3.

The extracted sample may be separated from the remaining components of the composition using standard techniques known to one of skill in the art. In certain embodiments, centrifugation may be performed. In certain embodiments, filtering the extractant may be performed. As a non-limiting example, filtering the extractant through a filter having a pore size of about 2 μm, or less, may be accomplished using standard techniques known to one of skill in the art. The resulting filtrate is referred to herein as a first filtrate.

The first filtrate may then be mixed with a precipitation reagent to produce precipitated calcium oxalate and a liquid solution. In preferred embodiments, the precipitation reagent comprises an oxalate salt. The amount or concentration of the oxalate salt may vary. In certain embodiments, the oxalate salt is present in an amount from about 0.10 to about 1.0 M, from about 0.10 to about 0.75 M, from about 0.10 to about 0.5 M, from about 0.15 to about 0.35 M, from about 0.28 to about 0.32, or about 0.3 M. In certain embodiments, the precipitation reagent has a pH of about 7.0, 7.1, or 7.2 or more.

The type of oxalate salt may vary. In certain embodiments, the oxalate salt may be selected from sodium oxalate, potassium oxalate, tin oxalate, naftidrofuryl oxalate, ammonium oxalate, escitalopram oxalate, cesium oxalate, lanthanum oxalate, cerium oxalate, and a mixture thereof. In certain embodiments, the oxalate salt is potassium oxalate. In certain embodiments, the oxalate salt is potassium oxalate monohydrate.

The precipitation reagent, and certain other reagents, of the present invention may comprise a buffer. In certain embodiments, the buffer has a pH of about 7.0, 7.1, or 7.2 or more. In certain embodiments, the buffer has a pH from about 7.0 to about 8.5, from about 7.0 to about 8.0, from about 7.1 to about 7.8, from about 7.18 to about 7.4, or about 7.2. In certain embodiments, the buffer is substantially free of, or is free of, calcium ions. In certain embodiments, the buffer comprises boric acid, tetrabutyl ammonium hydroxide, a carbonate, a phosphate, or a mixture thereof. In certain embodiments, the buffer comprises boric acid and tetrabutyl ammonium hydroxide. In certain embodiments, the buffer components, which are non-oxalate components, are present in an amount from about 0.10 to about 0.75 M, from about 0.10 to about 0.5 M, from about 0.10 to about 0.4 M, from about 0.15 to about 0.35 M, or about 0.3 M.

The volume ratio of first filtrate to precipitation reagent used to produce the precipitated calcium oxalate and a liquid solution may vary. In certain embodiments, the volume ratio of first filtrate to precipitation reagent is from about 20:1 to about 3:1, from about 20:1 to about 5:1, from about 17:1 to about 6:1, from about 15:1 to about 7:1, or about 9:1.

The precipitated calcium oxalate may be separated from the liquid solution of the composition by using techniques known to one of skill in the art. As a non-limiting example, separating the precipitated calcium oxalate from the liquid solution may be accomplished by use of filtration. In certain embodiments, a filter having a pore size of about 2 μm, or less, may be used using standard techniques known to one of skill in the art. In certain embodiments, a 1 μm filter may be used. In other embodiments, centrifugation may be used. In further embodiments, centrifugation and filtration may be used. In other embodiments, centrifugation may be used. The resulting filtrate may be referred to herein as a second filtrate.

The resulting separation step produces a composition which is substantially free of, or free of, calcium ions.

In certain embodiments, the invention is directed to a method for quantifying the magnesium ion amount in a soil sample. The method includes starting with a soil sample which is substantially free of, or free of, calcium ions. In certain embodiments, the methods and compositions described above may be used to produce a soil composition which is substantially free of, or free of, calcium ions. In other embodiments, other methods may be used to produce a soil composition which is substantially free of, or free of, calcium ions may be used. The soil composition, which is substantially free of, or free of, calcium ions may then be mixed with ethylene glycol-bis(β-aminoethyl ether)-N,N,N′,N′-tetraacetic acid tetrasodium salt (EGTA) to produce a first sample. The first sample may then be mixed with an indicator reagent to produce a second sample. In certain embodiments, the indicator reagent comprises o-cresolphthalein complexone (OCPC) and tetrabutyl ammonium hydroxide (TBAH). Finally, the magnesium ion amount, or concentration, of the second sample may be determined.

The EGTA may be used at various amounts or concentrations. In some embodiments, EGTA is present in an amount from about 0.1 to about 5 mM, from about 0.1 to about 3 mM, from about 0.1 to about 1.25 mM, from about 0.125 to about 1.25 mM, from about 0.125 to about 0.3 mM, or about 0.15 mM. In some embodiments, volume ratio of the substantially free, or free, calcium soil sample to EGTA used to produce a first sample is from about 1:2.5 to about 1:8, about 1:3 to about 1:7, from about 1:3 to about 1:5, or about 1:4.

The EGTA may be used with various buffers and pH values. In certain embodiments, the EGTA is within a buffer having a pH from about 8 to about 12, from about 9 to about 11, about 9.5 to about 10.5, or about 10. The buffer used may vary; however, it is preferred that the buffer is substantially free of, or is free of, calcium ions. In certain embodiments, the buffer comprises boric acid, an inorganic chloride salt (such as, but not limited to, sodium chloride, potassium chloride, and mixtures thereof), and sodium hydroxide. In certain embodiments, the buffer comprises boric acid, potassium chloride, and sodium hydroxide.

The indicator reagent may vary in its concentration and composition. In certain embodiments, the indicator reagent comprises o-cresolphthalein complexone (OCPC) and tetrabutyl ammonium hydroxide (TBAH). In certain embodiments, the OCPC is present within the indicator reagent in an amount from about 0.05 to about 0.5 mM, from about 0.06 to about 0.3 mM, from about 0.08 to about 0.16 mM, from about 0.1 to about 0.14 mM, or about 0.12 mM. In certain embodiments, the TBAH is present within the indicator reagent in an amount from about 0.005 to about 0.05 M, from about 0.006 to about 0.1 M, from about 0.008 to about 0.06 M, from about 0.01 to about 0.02 M, or about 0.015 M. In some embodiments, the volume ratio of the first sample to indicator reagent used to produce a second sample is from about is from about 1:2.5 to about 1:8, about 1:3 to about 1:7, from about 1:3 to about 1:5, or about 1:4.

The indicator reagent may be used with various buffers and pH values. In certain embodiments, the indicator reagent includes a buffer having a pH from about 8 to about 12, from about 9 to about 11, about 9.5 to about 10.5, or about 10. The buffer used may vary; however, it is preferred that the buffer is substantially free of, or is free of, calcium ions. In certain embodiments, the buffer comprises boric acid, an inorganic chloride salt (such as, but not limited to, sodium chloride, potassium chloride, and mixtures thereof), and sodium hydroxide. In certain embodiments, the buffer comprises boric acid, potassium chloride, and sodium hydroxide.

After producing the second sample, the quantitative determination of magnesium ion may be determined. In certain embodiments, the quantitative determination is made using spectroscopy. One of skill in the art would recognize that methods using spectroscopy include utilization of a calibration curve. The determination may be used at various wavelengths. In certain embodiments, the determination uses a wavelength from about 540 to about 585 nm, from about 555 to about 580 nm, or from about 560 to about 575 nm. In certain embodiments, the determination uses a wavelength of about 574 nm.

In certain embodiments, the present invention includes use of a system to carry out the methods as described herein. Various systems for soil collection, preparation and analysis may be used. For example, the compositions and methods described herein may be usable with and may form part of an overall agricultural sampling and analysis system, such as but not limited to those described in U.S. Patent Application Publication No. 2018/0124992A1 and PCT Publication No. WO2020/012369, and other systems are described in U.S. Application Nos. 62/983,237, filed on 28 Feb. 2020; 63/017,789, filed on 30 Apr. 2020; 63/017,840, filed on 30 Apr. 2020; 63/018,120, filed on 30 Apr. 2020; 63/018,153, filed on 30 Apr. 2020; 63/191,159, filed on 20 May 2021; 63/191,166, filed on 20 May 2021; 63/191,172, filed on 20 May 2021; Ser. No. 17/326,050, filed on 20 May 2021; 63/191,186, filed on 20 May 2021; 63/191,189, filed on 20 May 2021; 63/191,195, filed on 20 May 2021; 63/191,199, filed on 20 May 2021; 63/191,204, filed on 20 May 2021; Ser. No. 17/343,434, filed on 9 Jun. 2021; 63/208,865, filed on 9 Jun. 2021; Ser. No. 17/343,536, filed on 9 Jun. 2021; 63/213,319, filed on 22 Jun. 2021; 63/260,772 filed on 31 Aug. 2021; 63/260,776 filed on 31 Aug. 2021; 63/260,777 filed on 31 Aug. 2021, 63/245,278 filed on 17 Sep. 2021; 63/264,059 filed on 15 Nov. 2021; 63/264,062 filed on 15 Nov. 2021; 63/264,065 filed on 15 Nov. 2021; 63/268,418 filed on 23 Feb. 2022; 63/268,419 filed on 23 Feb. 2022; and 63/268,990 filed 8 Mar. 2022; and PCT Application Nos. PCT/IB2021/051076, filed on 10 Feb. 2021; PCT/IB2021/051077, filed on 10 Feb. 2021; PCT/IB2021/052872, filed on 7 Apr. 2021; PCT/IB2021/052874, filed on 7 Apr. 2021; PCT/IB2021/052875, filed on 7 Apr. 2021; and PCT/IB2021/052876, filed on 7 Apr. 2021.

In certain embodiments, the system is capable of extracting calcium and/or magnesium ions from a soil sample. In other embodiments, the system is capable of quantifying the magnesium ion amount in a soil sample. In further embodiments, the system is capable of extracting calcium and/or magnesium ions from a soil sample and then quantifying the magnesium ion amount in the soil sample. As a non-limiting example, the system may include a sample preparation sub-system which receives soil samples collected by a probe collection sub-system and produces a slurry (i.e. mixture of soil, vegetation, and/or manure and water) for further processing and chemical analysis, and a chemical analysis sub-system which receives and processes the prepared slurry samples from the sample preparation sub-system for quantification of the analytes and/or chemical properties of the sample. The chemical analysis sub-system can be used to analyze soil, vegetation, and/or manure samples. In certain embodiments, the sample preparation sub-system generally includes a mixer-filter apparatus which mixes the collected raw soil sample in the “as sampled” condition (e.g. undried and unground) with water to form a sample slurry. The mixer-filter apparatus may then further mix the slurry with a solution comprising ammonium acetate to produce an extracted sample. The mixer-filter apparatus then may filter the extracted sample to produce a first filtrate. The mixer-filter apparatus may then mix the first filtrate with a precipitation reagent to produce precipitated calcium oxalate and a liquid solution. Finally, the sample preparation sub-system may filter the calcium oxalate and a liquid solution to separate the two. The chemical analysis sub-system may further process the liquid solution (which is substantially free of, or free of, calcium ions) and performs the general functions of chelating to produce a first sample, mixing with indicator reagent, and finally sensing or analysis for detection of the analytes and/or chemical properties, such as via colorimetric analysis using a spectrometer. The system may further include a central processing unit (CPU) which comprises a system controller which may include one or more processors, non-transitory tangible computer readable medium, programmable input/output peripherals, and all other necessary electronic appurtenances normally associated with a fully functional processor based controller.

In other aspects of the invention, the compositions and methods described herein may be comprise a kit. As used herein, the term “kit” refers to any packaging or delivery system for delivering materials which enable one to practice the invention. In the context of reaction chemistry (such as when mixing components), such delivery systems include systems that allow for the storage, transport, or delivery of reaction reagents (e.g., ammonium acetate, precipitation reagent, indicator reagent, etc. in the appropriate containers) and/or supporting materials (e.g., filter, written instructions for performing the method etc.) from one location to another. For example, kits can include one or more enclosures (e.g., boxes) containing the relevant reaction reagents and/or supporting materials. The term “fragmented kit” may refer to a delivery system comprising two or more separate containers that each contains a sub-portion of the total kit components. The containers may be delivered to the intended recipient together or separately. For example, a first container may contain precipitation reagent, while a second container may contain indicator reagent. Indeed, any delivery system comprising two or more separate containers that each contains a sub-portion of the total kit components are included in the term “fragmented kit.” In contrast, a “combined kit” refers to a delivery system containing all of the components required to perform the method in a single container (e.g., in a single box housing each of the desired components). The term “kit” includes both fragmented and combined kits.

### Examples

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

Example A: Precipitation and separation of calcium ions from soil extract. Calcium ions from soil slurry were extracted using a solution having about 1M ammonium acetate in water as an extractant reagent in addition to use of a precipitation reagent (pH of about 7.2) as described in Table 1. The precipitation reagent was tested using either water, ethanol, or isopropanol as a solvent.

TABLE 1
Precipitation reagent composition.

| Component | Concentration (M) |
|---|---|
| Potassium oxalate monohydrate | 0.3 |
| Boric acid/Tetrabutyl Ammonium hydroxide | 0.3 |

Calcium extraction was initiated by mixing water with the soil in ratios from about 1:2.5 to about 1:3.5 soil to water ratio, or about a 1:3 soil to water, to make a soil slurry. The soil slurry samples were then mixed with extractant reagent to achieve from about a 1:1 to about a 1:5 ratio of slurry to extractant reagent, or about a 1:3 ratio of slurry to extractant reagent. The mixture of slurry and extractant reagent was then filtered using a pore size from about 0.2 μm to about 2 μm and a precipitation reaction with potassium oxalate was used to remove calcium ions from the filtrate. To achieve precipitation, the filtrate was mixed with the precipitation reagent at a volume ratio of between about 5% to about 20% (0.05:1 to about 0.2:1), or about 10% (0.1:1), of the precipitation reagent to filtrate. The filtrate and precipitation reagent were generally allowed to mix gently for about one to six minutes. Generally, two to three minutes was sufficient to remove nearly 99% of calcium from the filtrate solution. More so, the precipitation reaction was optimal when the pH of the mixed filtrate/precipitation reagent was higher than 7.0.

When the precipitation of calcium ions was completed, solid calcium oxalate was removed from the solution by filtration using a pore size of about 2 μm or less or by centrifuging the samples from about 3300 rpm to about 9200 rpm for about 2 minutes or less to produce a final filtrate. Significantly, use of a 5 μm filter allowed some calcium oxalate particles to pass through. Therefore, use of smaller filters, such as a 2 μm filter or less, is preferred.

In soil samples having low parts per million (ppm) calcium ion concentration, additional use of a rough surface material enhanced calcium precipitation. For example, the addition of a seed crystal having a rough surface, such as addition of sand or glass beads, and/or use of a container having rough surface may be used. Surprisingly, the additional use of a rough surface material increased the calcium precipitation by over 20% in soil samples having low ppm calcium ion.

Regarding solvent testing for the precipitation reagent, the use of water within the precipitation reagent provided for optimal results. When using ethanol, the buffered oxalate salt reagent was soluble in a solution composed of 50% ethanol and 50% water. Unexpectedly, while the reagent effected to precipitate calcium ion from the solution, it also caused some magnesium oxalate to precipitate out of solution as well. As such, precipitation reagent having up to 50% ethanol in water may be used. By contrast, presence of isopropanol greatly reduced the solubility of the chemical components in the reagent. Even for solutions having less than 30% isopropanol present, the required concentrations of the solution components could not be achieved.

Example B: Precipitation and quantitative determination of magnesium ions from soil extract. Magnesium ions from soil extract were prepared for analysis using extractant reagent (as described above) in addition to use of a chelation reagent (about 0.15 mM ethylene glycol-bis(β-aminoethyl ether)-N,N,N′,N′-tetraacetic acid tetrasodium salt (EGTA) in buffer reagent at pH 10), indicator reagent (about 0.12 mM o-cresolphthalein complexone (OCPC) and about 0.015M tetrabutyl ammonium hydroxide (TBAH) in buffer reagent at pH 10), where the buffer reagent (pH of about 10.0) is as described in Table 2. Use of either water, ethanol, or isopropanol was tested as a solvent for the buffer reagent.

TABLE 2
Buffer reagent composition.

| Component | Concentration (M) |
|---|---|
| Boric acid | 0.3-0.5 |
| Potassium chloride | 0.06-0.1 |
| Sodium hydroxide | 0.22-0.38 |

About a 0.250 mL aliquot of the final filtrate (or supernatant if centrifugation was used) from Example 1 was mixed with about 1 ml of the chelation reagent (about a 1:5 dilution). About a 0.250 mL aliquot of the mixed final filtrate/chelation reagent composition was then mixed with about 1 ml of the indicator reagent (about a 1:5 dilution) and the absorbance of the sample was measured at between 540 to 585 nm, from 555 to 580 nm, or about 574 nm.

Quantitative determination of magnesium ion concentration was made by comparison of the absorbance of the test sample to absorbance of standards. Calibration curves were made by preparing from one to about six standards of magnesium in nitric acid and ammonium acetate, where the standard compositions had from about 0 to about 480 ppm magnesium ion. The regression equation for the calibration curve was created using either a linear, binomial, or a trinomial curve fit.

When using water as a solvent for the buffer reagent, optimal results were achieved. Use of 50% ethanol in 50% water resulted in the calibration curve with a binomial curve fit. Also, the presence of ethanol reduced the observed color of the indication reagent and reduced the buffer capacity of the reagents. As a result, the colorimetric test solutions for the standards had higher pH compared to the soil sample test solutions and yielded lower than expected magnesium values for the soils. However, the calculated results were within 20% of magnesium values as determined in parallel by use of Inductively Coupled Plasma Spectroscopy, or ICP method. Nonetheless, use of solutions containing up to 50% ethanol in water could be used as a solvent for the buffer reagent composition. By contrast, use of isopropanol as a solvent within the buffer reagent provided for poor solubility of the chemical components.

Example 1—a method of extracting calcium ions from soil comprising: mixing soil with a solvent to produce a soil slurry; mixing the soil slurry with ammonium acetate to produce an extracted sample; filtering the extracted sample to produce a first filtrate; mixing the first filtrate with a precipitation reagent to produce precipitated calcium oxalate and a liquid solution; and separating the calcium oxalate from the liquid solution; wherein the precipitation reagent comprises from about 0.15 to about 0.35 M of an oxalate salt dissolved in a buffer having a pH from about 7.1 or more.

Example 2—the method of Example 1, wherein the solvent is water.

Example 3—the method of Example 1 or 2, wherein the volume ratio of soil to solvent used to produce the soil slurry is from about 1:2.5 to about 1:3.5 or from about 1:2.9 to about 1:3.1.

Example 4—the method of any one of the preceding Examples, wherein the concentration of ammonium acetate is from about 0.5 to about 1.5 M, from about 0.6 to about 1.4 M, or from about 0.75 to about 1.25 M.

Example 5—the method of any one of the preceding Examples, wherein the volume ratio of soil slurry to ammonium acetate used to produce the extracted sample is from about 1:1 to about 1:5 or from about 1:2 to about 1:4.

Example 6—the method of any one of the preceding Examples, wherein the volume ratio of first filtrate to precipitation reagent used to produce the precipitated calcium oxalate and a liquid solution is from about 20:1 to about 5:1 or from about 15:1 to about 7:1.

Example 7—the method of any one of the preceding Examples, wherein the oxalate salt is selected from sodium oxalate, potassium oxalate, tin oxalate, naftidrofuryl oxalate, ammonium oxalate, escitalopram oxalate, cesium oxalate, lanthanum oxalate, cerium oxalate, and a mixture thereof.

Example 8—the method of any one of the preceding Examples, wherein the concentration of the oxalate salt is from about 0.28 to about 0.32 or about 0.3 M.

Example 9—the method of any one of the preceding Examples, wherein the oxalate salt is dissolved in buffer comprising boric acid, tetrabutyl ammonium hydroxide, a carbonate, a phosphate, or a mixture thereof.

Example 10—the method of any one of the preceding Examples, wherein the oxalate salt is dissolved in buffer comprising boric acid and tetrabutyl ammonium hydroxide.

Example 11—the method of Example 8 or 9, wherein the concentration of non-oxalate components is from about 0.15 to about 0.35 M.

Example 12—the method of any one of Examples 9 to 11, wherein the pH of the buffer is from about 7.1 to about 8.0 or from about 7.18 to about 7.4.

Example 13—the method of any one of the preceding Examples, wherein the separation of calcium oxalate from liquid solution is performed by filtration.

Example 14—the method of Example 13, wherein the filtration is performed using a filter having a pore diameter of less than about 2 μm.

Example 15—the method of Example 13, wherein the filtration is performed using a filter having a pore diameter of about 1 μm or less.

Example 16—the method of any one of Examples 1 to 12, wherein the separation of calcium oxalate from liquid solution is performed by centrifugation.

Example 16—the method of any one of the preceding Examples, wherein the separation of calcium oxalate from liquid solution is performed by centrifugation and filtration.

Example 18—a system capable of extracting calcium ions from soil using any one of the methods of Examples 1 to 17, the system comprising: means to mix soil and solvent to produce a soil slurry; means to mix soil slurry with ammonium acetate to produce an extracted sample; means to filter the extracted sample to produce a first filtrate; means to mix the first filtrate with a precipitation reagent to produce precipitated calcium oxalate and a liquid solution; and means to separate the calcium oxalate from the liquid solution.

Example 19—a kit capable of extracting calcium ions from soil using any one of the methods of Examples 1 to 17, the system comprising: means to mix soil and solvent to produce a soil slurry; means to mix soil slurry with ammonium acetate to produce an extracted sample; means to filter the extracted sample to produce a first filtrate; means to mix the first filtrate with a precipitation reagent to produce precipitated calcium oxalate and a liquid solution; and means to separate the calcium oxalate from the liquid solution.

Example 20—a method for quantifying the magnesium ion amount in a soil sample, the method comprising: extracting calcium ions from a soil sample to produce a soil sample substantially free of calcium; mixing the substantially free calcium soil sample with ethylene glycol-bis(β-aminoethyl ether)-N,N,N′,N′-tetraacetic acid tetrasodium salt (EGTA) to produce a first sample; mixing the first sample with an indicator reagent to produce a second sample; and determining the magnesium ion amount of the second sample; wherein the indicator reagent comprises o-cresolphthalein complexone (OCPC) and tetrabutyl ammonium hydroxide (TBAH).

Example 21—the method of Example 20, wherein the EGTA has a concentration from about 0.125 to about 1.25 mM or from about 0.125 to about 0.3 mM.

Example 22—the method of Example 20 or 21, wherein EGTA is in a buffered solution at a pH from about 9 to about 11, about 9.5 to about 10.5, or about 10.

Example 23—the method of any one of Examples 20 to 22, wherein EGTA is in a buffer comprising boric acid, sodium hydroxide, and a salt selected from potassium chloride, sodium chloride, and a mixture thereof.

Example 24—the method of Example 23, wherein the salt is potassium chloride.

Example 25—the method of any one of Examples 20 to 24, wherein the volume ratio of the substantially free calcium soil sample to EGTA to produce a first sample is from about 1:3 to about 1:7 or from about 1:3 to about 1:5.

Example 26—the method of any one of Examples 20 to 25, wherein the OCPC is present within the indicator reagent at a concentration from about 0.08 to about 0.16 mM or from about 0.1 to about 0.14 mM.

Example 27—the method of any one of Examples 20 to 26, wherein the TBAH is present within the indicator reagent at a concentration from about 0.008 to about 0.06 M or from about 0.01 to about 0.02 M.

Example 28—the method of any one of Examples 20 to 27, wherein the pH of the indicator reagent is about 10.

Example 29—the method of any one of Examples 20 to 28, wherein the indicator reagent comprises boric acid, sodium hydroxide, and a salt selected from potassium chloride, sodium chloride, and a mixture thereof.

Example 30—the method of Example 29, wherein the salt is potassium chloride.

Example 31—the method of any one of Examples 20 to 30, wherein the volume ratio of the first sample to indicator reagent to produce a second sample is from about 1:3 to about 1:7 or from about 1:3 to about 1:5.

Example 32—the method of any one of Examples 20 to 31, wherein the magnesium ion determination is performed using spectrophotometry.

Example 33—the method of any one of Examples 20 to 31, wherein the determination uses a wavelength from 540 to 585 nm, from 555 to 580 nm, or from 560 to 575 nm.

Example 34—the method of any one of Examples 20 to 32, wherein the determination uses a wavelength of about 574 nm.

Example 35—the method of Example 20, wherein the calcium ion extraction is performed by comprising the steps of: mixing soil with a solvent to produce a soil slurry; mixing the soil slurry with ammonium acetate to produce an extracted sample; filtering the extracted sample to produce a first filtrate; mixing the first filtrate with a precipitation reagent to produce precipitated calcium oxalate and a liquid solution; and separating the calcium oxalate from the liquid solution; wherein the precipitation reagent comprises from about 0.15 to about 0.35 M of an oxalate salt dissolved in a buffer having a pH from about 7.1 or more.

Example 36—the method of Example 35, wherein the solvent is water.

Example 37—the method of Example 35 or 36, wherein the volume ratio of soil to solvent used to produce the soil slurry is from about 1:2.5 to about 1:3.5 or from about 1:2.9 to about 1:3.1.

Example 38—the method of any one of Examples 35 to 37, wherein the concentration of ammonium acetate is from about 0.5 to about 1.5 M, from about 0.6 to about 1.4 M, or from about 0.75 to about 1.25 M.

Example 39—the method of any one of Examples 35 to 38, wherein the volume ratio of soil slurry to ammonium acetate used to produce the extracted sample is from about 1:1 to about 1:5 or from about 1:2 to about 1:4.

Example 40—the method of any one of Examples 35 to 39, wherein the volume ratio of first filtrate to precipitation reagent used to produce the precipitated calcium oxalate and a liquid solution is from about 20:1 to about 5:1 or from about 15:1 to about 7:1.

Example 41—the method of any one of Examples 35 to 40, wherein the oxalate salt is selected from sodium oxalate, potassium oxalate, tin oxalate, naftidrofuryl oxalate, ammonium oxalate, escitalopram oxalate, cesium oxalate, lanthanum oxalate, cerium oxalate, and a mixture thereof.

Example 42—the method of any one of Examples 35 to 41, wherein the concentration of the oxalate salt is from about 0.28 to about 0.32 or about 0.3 M.

Example 43—the method of any one of Examples 35 to 42, wherein the oxalate salt is dissolved in buffer comprising boric acid, tetrabutyl ammonium hydroxide, a carbonate, a phosphate, or a mixture thereof.

Example 44—the method of any one of Examples 35 to 43, wherein the oxalate salt is dissolved in buffer comprising boric acid and tetrabutyl ammonium hydroxide.

Example 45—the method of Example 42 or 44, wherein the concentration of non-oxalate components is from about 0.15 to about 0.35 M.

Example 46—the method of any one of Examples 35 to 45, wherein the pH of the buffer is from about 7.1 to about 8.0 or from about 7.18 to about 7.4.

Example 47—the method of any one of Examples 35 to 46, wherein the separation of calcium oxalate from liquid solution is performed by filtration.

Example 48—the method of Example 47, wherein the filtration is performed using a filter having a pore diameter of less than about 2 μm.

Example 49—the method of Example 47, wherein the filtration is performed using a filter having a pore diameter of about 1 μm or less.

Example 50—the method of any one of Examples 35 to 49, wherein the separation of calcium oxalate from liquid solution is performed by centrifugation.

Example 51—the method of any one of Examples 35 to 50, wherein the separation of calcium oxalate from liquid solution is performed by centrifugation and filtration.

Example 52—a system capable of quantifying the magnesium ion amount in a soil sample using any one of the methods of Examples 20 to 51, the system comprising: means to obtain a soil slurry substantially free from calcium ions; means to mix the soil slurry with EGTA to produce a first sample; means to mix the first sample with an indicator reagent to produce a second sample; and means to determine the magnesium ion amount of the second sample.

Example 53—a kit capable of quantifying the magnesium ion amount in a soil sample using any one of the methods of Example 20 to 51, the system comprising: means to obtain a soil slurry substantially free from calcium ions; means to mix the soil slurry with EGTA to produce a first sample; means to mix the first sample with an indicator reagent to produce a second sample; and means to determine the magnesium ion amount of the second sample.

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

1. A method for quantifying the magnesium ion amount in a soil sample, the method comprising:
a) extracting calcium ions from a soil sample to produce a soil sample substantially free of calcium;
b) mixing the substantially free calcium soil sample with ethylene glycol-bis(β-aminoethyl ether)-N,N,N′,N′-tetraacetic acid tetrasodium salt (EGTA) to produce a first sample;
c) mixing the first sample with an indicator reagent to produce a second sample; and
d) determining the magnesium ion amount of the second sample;
wherein the indicator reagent comprises o-cresolphthalein complexone (OCPC) and tetrabutyl ammonium hydroxide (TBAH).

2. The method of claim 1, wherein the EGTA has a concentration from about 0.125 to about 1.25 mM or from about 0.125 to about 0.3 mM.

3. The method of claim 1, wherein EGTA is in a buffered solution at a pH from about 9 to about 11, about 9.5 to about 10.5, or about 10.

4. The method of claim 1, wherein EGTA is in a buffer comprising boric acid, sodium hydroxide, and a salt selected from potassium chloride, sodium chloride, and a mixture thereof.

5. The method of claim 4, wherein the salt is potassium chloride.

6. The method of claim 1, wherein the volume ratio of the substantially free calcium soil sample to EGTA to produce a first sample is from about 1:3 to about 1:7 or from about 1:3 to about 1:5.

7. The method of claim 1, wherein the OCPC is present within the indicator reagent at a concentration from about 0.08 to about 0.16 mM or from about 0.1 to about 0.14 mM.

8. The method of claim 1, wherein the TBAH is present within the indicator reagent at a concentration from about 0.008 to about 0.06 M or from about 0.01 to about 0.02 M.

9. The method of claim 1, wherein the pH of the indicator reagent is about 10.

10. The method of claim 1, wherein the indicator reagent comprises boric acid, sodium hydroxide, and a salt selected from potassium chloride, sodium chloride, and a mixture thereof.

11. The method of claim 10, wherein the salt is potassium chloride.

12. The method of claim 1, wherein the volume ratio of the first sample to indicator reagent to produce a second sample is from about 1:3 to about 1:7 or from about 1:3 to about 1:5.

13. The method of claim 1, wherein the magnesium ion determination is performed using spectrophotometry.

14. The method of claim 1, wherein the determination uses a wavelength from 540 to 585 nm, from 555 to 580 nm, or from 560 to 575 nm.

15. The method of claim 1, wherein the determination uses a wavelength of about 574 nm.

16. The method of claim 1, wherein the calcium ion extraction is performed by comprising the steps of:
a) mixing soil with a solvent to produce a soil slurry;
b) mixing the soil slurry with ammonium acetate to produce an extracted sample;
c) filtering the extracted sample to produce a first filtrate;
d) mixing the first filtrate with a precipitation reagent to produce precipitated calcium oxalate and a liquid solution; and
e) separating the calcium oxalate from the liquid solution;
wherein the precipitation reagent comprises from about 0.15 to about 0.35 M of an oxalate salt dissolved in a buffer having a pH from about 7.1 or more.

17. The method of claim 16, wherein the solvent is water.

18. The method of claim 16, wherein the volume ratio of soil to solvent used to produce the soil slurry is from about 1:2.5 to about 1:3.5 or from about 1:2.9 to about 1:3.1.

19. The method of claim 16, wherein the concentration of ammonium acetate is from about 0.5 to about 1.5 M, from about 0.6 to about 1.4 M, or from about 0.75 to about 1.25 M.

20. The method of claim 16, wherein the volume ratio of soil slurry to ammonium acetate used to produce the extracted sample is from about 1:1 to about 1:5 or from about 1:2 to about 1:4.

21. The method of claim 16, wherein the volume ratio of first filtrate to precipitation reagent used to produce the precipitated calcium oxalate and a liquid solution is from about 20:1 to about 5:1 or from about 15:1 to about 7:1.

22. The method of claim 16, wherein the oxalate salt is selected from sodium oxalate, potassium oxalate, tin oxalate, naftidrofuryl oxalate, ammonium oxalate, escitalopram oxalate, cesium oxalate, lanthanum oxalate, cerium oxalate, and a mixture thereof.

23. The method of claim 16, wherein the concentration of the oxalate salt is from about 0.28 to about 0.32 or about 0.3 M.

24. The method of claim 16, wherein the oxalate salt is dissolved in buffer comprising boric acid, tetrabutyl ammonium hydroxide, a carbonate, a phosphate, or a mixture thereof.

25. The method of claim 16, wherein the oxalate salt is dissolved in buffer comprising boric acid and tetrabutyl ammonium hydroxide.

26. The method of claim 23, wherein the concentration of non-oxalate components is from about 0.15 to about 0.35 M.

27. The method of claim 16, wherein the pH of the buffer is from about 7.1 to about 8.0 or from about 7.18 to about 7.4.

28. The method of claim 16, wherein the separation of calcium oxalate from liquid solution is performed by filtration.

29. The method of claim 28, wherein the filtration is performed using a filter having a pore diameter of less than about 2 μm.

30. The method of claim 28, wherein the filtration is performed using a filter having a pore diameter of about 1 μm or less.

31. The method of claim 16, wherein the separation of calcium oxalate from liquid solution is performed by centrifugation.

32. The method of claim 16, wherein the separation of calcium oxalate from liquid solution is performed by centrifugation and filtration.

33. A system capable of quantifying the magnesium ion amount in a soil sample using claim 1, the system comprising:
means to obtain a soil slurry substantially free from calcium ions;
means to mix the soil slurry with EGTA to produce a first sample;
means to mix the first sample with an indicator reagent to produce a second sample; and
means to determine the magnesium ion amount of the second sample.

34. A kit capable of quantifying the magnesium ion amount in a soil sample using claim 1, the system comprising:
means to obtain a soil slurry substantially free from calcium ions;
means to mix the soil slurry with EGTA to produce a first sample;
means to mix the first sample with an indicator reagent to produce a second sample; and
means to determine the magnesium ion amount of the second sample.
